(19) United States
(12) Patent Application Publication
(10) Pub. No.: US 2004/0202681 A1
(43) Pub. Date: Oct. 14, 2004

(54) Process for preparing pharmaceutical formulations using supercritical fluids

(75) Inventors: Satish Pejaver, Bridgewater, NJ (US); Navneet Puri, Bridgewater, NJ (US); Poongunran Muthukumaran, Pittsburgh, PA (US); Lalit Chordia, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignees: BAXTER INTERNATIONAL, INC., Deerfield, IL; THAR TECHNOLOGIES, INC., Pittsburgh, PA

(21) Appl. No.: 10/741,225

(22) Filed: Dec. 19, 2003

Related U.S. Application Data
(60) Provisional application No. 60/435,054, filed on Dec. 19, 2002.

(51) Int. Cl. A61K 38/16; A61K 33/24; A61K 9/00

(52) U.S. Cl. 424/400; 424/649; 514/192; 514/35; 514/34; 514/50; 514/8; 514/283

(57) ABSTRACT

The invention is directed to a process for preparing a pharmaceutical formulation containing two or more active pharmaceutical ingredients comprising: (a) contacting two or more active pharmaceutical ingredients with a supercritical fluid to form a supercritical fluid solution; and (b) separating the active ingredients from the supercritical solution to yield a powder precipitate. Preferably, the pharmaceutical formulation prepared according to the invention contains a combination of two anti-infective agents or two anticancer agents. The invention is further directed to a process for preparing a pharmaceutical formulation containing two or more active pharmaceutical ingredients comprising: (a) combining two or more active ingredients with a cosolvent to form a solution; (b) contacting the solution with a supercritical fluid; and (c) recovering the precipitate in a powder form.

### CROSS-REFERENCE TO RELATED PATENT APPLICATIONS

This patent application claims the benefit of U.S. Provisional Patent Application No. 60/435,054, filed Dec. 19, 2002.

### FIELD OF THE INVENTION

This invention pertains to a process for combining two or more active pharmaceutical ingredients including, for example, anti-infective and anticancer agents, using a supercritical fluid to obtain a blended, dry powder pharmaceutical formulation.

### BACKGROUND OF THE INVENTION

Pharmaceuticals containing a combination of two or more active pharmaceutical ingredients, especially anti-infective agents, are commercially available in a dry powder form. Anti-infective agents as well as many other active agents are not stable for extended periods of time in an aqueous solution which requires the preparation of such actives as a solid powder.

Combination anti-infectives are typically produced by milling of the active agents and excipients and blending the dry solid components to form the finished drug product. However, the use of milling and blending techniques has several significant limitations. Most significantly, the mechanical equipment used to accomplish the milling and blending operations is in direct contact with the drug product components which can result in contamination from pyrogens and/or particular matter. Such contaminants compromise the sterility required for pharmaceutical products that are administered parenterally. Other drawbacks include, for example, the need for specialized ventilation equipment to collect dust produced during milling, the difficulty in obtaining blend uniformity, and the degradation of the active ingredients and excipients caused by high shear milling. Moreover, the potential segregation of the components of the blended powder during its transfer from blender to the filling line and during vial filling may eventually lead to content non-uniformity in the final blended drug product.

An alternative to the use of traditional milling and blending procedures to produce combination drug products is spray drying. The spray drying process involves the dissolution of active agents in a suitable cosolvent (which may be a single solvent or two or more solvents combined together) followed by spraying of the solution in a heated chamber. However, spray drying has several significant limitations. Stability issues exist with the solution or dispersion of the active agents formed before spraying. In addition, the high temperatures used during the process can cause degradation of the drugs. Spray drying also gives low yields of the final product and often requires the use of a secondary drying step to ensure removal of cosolvent from the powder.

Thus, there remains a need for an efficient process for producing sterile combination pharmaceutical drug products in a powder form that exhibit good blend uniformity.

The invention provides such a process for preparing sterile pharmaceutical formulations in a dry powder form that contain two or more active pharmaceutical ingredients in a homogenous blend. These and other advantages of the invention, as well as additional inventive features, will be apparent from the description of the invention provided herein.

### BRIEF SUMMARY OF THE INVENTION

The invention provides a process for preparing a pharmaceutical formulation containing two or more active pharmaceutical ingredients by (a) contacting two or more active ingredients with a supercritical fluid, and (b) separating the active ingredients from the supercritical fluid to yield a dry powder precipitate containing the active ingredients. The invention further provides a supercritical fluid solution comprising a supercritical fluid and two or more active pharmaceutical ingredients.

The invention further relates to a process for preparing a pharmaceutical formulation containing a combination of two anti-infective agents comprising:

(a) contacting two anti-infective agents with supercritical carbon dioxide to form a supercritical carbon dioxide solution;

(b) spraying the supercritical carbon dioxide solution through a nozzle; and

(c) recovering the precipitate in a powder form containing the combination of anti-infective agents.

In another embodiment, the invention is directed to a process for preparing a pharmaceutical formulation containing two or more active pharmaceutical ingredients comprising:

(a) combining two or more active ingredients with a solvent to form a solution;

(b) contacting the solution with a supercritical fluid; and

(c) recovering the precipitate in a powder form.

The invention further includes a process for preparing a pharmaceutical formulation containing a combination of two anti-infective agents comprising:

(a) combining two anti-infective agents with a solvent to form a solution;

(b) contacting the solution with supercritical carbon dioxide; and

(c) recovering the precipitate in a powder form containing the combination of anti-infective agents.

In another embodiment, the present invention provides a process for preparing a combination product containing two or more substances comprising:

(a) contacting two or more desired substances with a supercritical fluid to form a supercritical fluid solution; and

(b) separating the substances from the supercritical fluid solution to yield a powder precipitate.

The invention is further directed to a process for preparing a combination product containing two or more substances comprising:

(a) combining two or more substances with a solvent to form a solution;

(b) mixing the solution with a supercritical fluid; and

(c) recovering the precipitate in a powder form.

### BRIEF DESCRIPTION OF THE DRAWINGS

FIG. 1 is a schematic of the apparatus for the recrystallization of pharmaceutical formulations containing two or more active pharmaceutical ingredients using the RESS technique.

FIG. 2 is a schematic of the apparatus for the recrystallization of pharmaceutical formulations containing two or more active pharmaceutical ingredients using the SAS technique.

FIG. 3 is a schematic of the apparatus for the recrystallization of pharmaceutical formulations containing two or more active pharmaceutical ingredients using the GAS technique.

FIG. 4 is a schematic of the apparatus for the recrystallization of pharmaceutical formulations according to Examples 1-2, 12, 18 and 20-21.

FIG. 5 is a schematic of the apparatus for the recrystallization of pharmaceutical formulations according to Example 19.

FIG. 6 is a schematic of the apparatus for the recrystallization of pharmaceutical formulations according to Examples 3-11 and 13-17.

### DETAILED DESCRIPTION OF THE INVENTION

The present invention is directed to a process for preparing pharmaceutical formulations containing two or more active pharmaceutical ingredients using supercritical fluid technology.

A “supercritical fluid” is a fluid at or above its critical pressure (P_{c}) and critical temperature (T_{c}) simultaneously. Thus, a fluid above its critical pressure and at its critical temperature is in a supercritical state. A fluid at its critical pressure and above its critical temperature is also supercritical. As used herein, an “antisolvent” is a supercritical fluid.

As used herein, supercritical fluids also encompass both near supercritical fluids and subcritical fluids. A “near supercritical fluid” is above but close to its critical pressure (P_{c}) and critical temperature (T_{c}) simultaneously. A “subcritical fluid” is above its critical pressure (P_{c}) and close to its critical temperature (T_{c}).

Any suitable supercritical fluid may be used in the process of the present invention. The supercritical fluid should be compatible with the active agents that are dissolved in or contacted with the supercritical fluid in the recrystallization processes detailed herein.

Typical supercritical fluids and their critical properties (i.e., critical temperature, critical pressure, and critical density) are listed in Table 1.

TABLE 1

| Fluid | T_{c} (° C.) | P_{c} (MPa) | ρ_{c} (g/cm³) |
|---|---|---|---|
| ethylene | 9.3 | 5.04 | 0.22 |
| xenon | 16.6 | 5.84 | 0.12 |
| carbon dioxide | 31.1 | 7.38 | 0.47 |
| ethane | 32.2 | 4.88 | 0.20 |
| nitrous oxide | 36.5 | 7.17 | 0.45 |
| propane | 96.7 | 4.25 | 0.22 |
| ammonia | 132.5 | 11.28 | 0.24 |
| n-butane | 152.1 | 3.80 | 0.23 |
| n-pentane | 196.5 | 3.37 | 0.24 |
| isopropanol | 235.2 | 4.76 | 0.27 |
| methanol | 239.5 | 8.10 | 0.27 |
| toluene | 318.6 | 4.11 | 0.29 |
| water | 374.2 | 22.05 | 0.32 |

Carbon dioxide is preferably utilized used as the supercritical fluid for producing pharmaceutical formulations containing two or more active agents according to the present invention. The use of supercritical carbon dioxide in pharmaceutical processing is further described in Subramaniam et al., J. Pharm. Sci. 1997: 86, 8, which is incorporated herein by reference.

Other suitable supercritical fluids, also referred to as antisolvents, useful in the present invention include water, ammonia, nitrogen, nitrous oxide, methane, ethane, ethylene, propane, butane, n-pentane, benzene, methanol, ethanol, isopropanol, 1-propanol, isobutanol, 1-butanol, monofluoromethane, trifluoromethane, chlorotrifluoromethane, monofluoromethane, hexafluoroethane, 1,1-difluoroethylene, 1,2-difluoroethylene,toluene, pyridine, cyclohexane, m-cresol, decalin, cyclohexanol, xylene, tetralin, aniline, acetylene, chlorotrifluorosilane, xenon, sulfur hexafluoride and combinations thereof.

Any combination of two or more active pharmaceutical ingredients may be used in the present invention. Preferably, two or more anti-infectives are combined in pharmaceutical formulations of the present invention. More preferably, two anti-infectives are combined.

Some examples of anti-infectives suitable for use including macrolide antibiotics such as clarithromycin, erythromycin, and azithromycin, anthracycline antibiotics such as doxorubicin and daunorubicin, camptothecin and its analogs such as topotecan and irenotecan, and quinolone antibiotics such as ciprofloxacin, ofloxacin, levofloxacin, clinafloxacin, and moxifloxacin. Cephalosporins may also be used such as, for example, cefotaxime, ceftriaxone, ceftazidime, and cefepime.

Other suitable anti-infective agents include β-lactam antibiotics (e.g., cefotetan, aztreonam), penicillins (e.g., amoxicillin, piperacillin), aminoglycosides (e.g., streptomycin), and sulfonamides (e.g., trimethoprim/sulfamethoxazole). Further anti-infective agents and classes thereof that may be used include, without limitation, carbapenems, bacitracin, gramicidin, mupirocin, chloramphenicol, thiamphenicol, fusidate sodium, lincomycin, clindamycin, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitroffurans, methenamine mandelate and methenamine hippurate, nitroimidazoles, fluoroquinolones, isoniazid, ethambutol, pyrazinamide, para-aminosalicylic acid (PAS), cycloserine, capreomycin, ethionamide, prothionamide, thiacetazone and viomycin. Specific anti-infectives that are suitable include, without limitation, amikacin, netilmicin, fosfomycin, gentamicin, and teicoplanin.

Preferably the anti-infectives useful in the present invention include ampicillin sodium, sulbactam sodium, ticarcillin disodium, clavulanate potassium, quinupristin, dalfopristin, piperacillin sodium, tazobactam, imipenem and cilastatin.

Most preferably, pharmaceutical drug products containing two anti-infective active ingredients are produced according to the invention. The following combinations of anti-infective agents are preferably used: ampicillin sodium/sulbactam sodium (marketed under the brand name Unasyn® by Pfizer); ticarcillin disodium/clavulanate potassium (marketed under the brand name Timentin® by GlaxoSmithKline); quinupristin/dalfopristin (marketed under the brand name Synercid® by Aventis); piperacillin sodium/tazobactam sodium (marketed under the brand name Zosyn® by Lederle Pharmaceutical); and, imipenem/cilastatin (marketed under the brand name Primaxin® by Merck).

In another embodiment, the present invention includes pharmaceutical drug products containing two or more anticancer agents. Preferably, pharmaceutical drug products containing two anticancer active ingredients are produced. The following anticancer agents are preferably used etoposide, paclitaxel, cisplatin, sarcolysine, alkylating agents, bleomycin, busulfan, docetaxel, carboplatin, doxorubicin, vincristine, fluorouracil, methotrexate, vinorelbine, cyclophosphamide, etoposide, ifosfamide, mesna, gemcitabine hydrochloride, irinotecan hydrochloride, 5-fluorouracil, platinoids, and vinorelbine tartarate.

More preferably, the following combinations of two anticancer agents are preferably used: etoposide/paclitaxel; altretamine/cisplatin; altretamine/sarcolysine; altretamine/alkylating agents; bleomycin/cisplatin; busulfan/docetaxel; busulfan/carboplatin; cisplatin/doxorubicin; cisplatin/vincristine; cisplatin/fluorouracil; cisplatin/methotrexate; cisplatin/vinorelbine; cyclophosphamide/etoposide; etoposide/ifosfamide; ifosfamide/mesna; gemcitabine hydrochloride/cisplatin; gemcitabine/paclitaxel; irinotecan hydrochloride/5-fluorouracil; paclitaxel/platinoids; vinorelbine tartarate/platinoids; vinorelabine tartrate/paclitaxel; paclitaxel/cisplatin; and toposide/cisplatin.

Other types of active pharmaceutical ingredients that may be combined according to the present invention include the following classes of drugs: anxiolytic (e.g., diazepam), antidepressant (e.g., fluoxetine), anesthetic (e.g., midazolam), antiviral (e.g., ganciclovir), protease inhibitor (e.g., saquinavir), chemotherapeutic (e.g., mesna, paclitaxel, cisplatin), anti-inflammatory (e.g., naproxen, ketorolac), antimalarial (e.g., mefloquine), antihypertensive (e.g., enalapril, lisinopril), antiseborheic (e.g., isotretinoin), calcium channel blocker (e.g., diltiazem, nifedipine), lipase inhibitor (e.g., orlistat), antiparkinson (e.g., tolcapone), antiarthritic (e.g., mycophenolate mofetil), and thrombolytic agent (e.g., streptokinase). Also contemplated within the scope of the present invention are additional classes of drugs that are administered to a patient to obtain a desired therapeutic effect.

The pharmaceutical ingredients useful in the present invention may be any known or hereafter discovered pharmacologically active ingredient, and may be a compound that occurs in nature, a chemically modified naturally occurring compound, or a compound that is chemically synthesized. The ingredient will typically be chosen from the generally recognized classes of pharmacologically active ingredients, including, but not necessarily limited to, the following: analgesic ingredients; anesthetic ingredients; antiarthritic ingredients; respiratory drugs, including antiasthmatic ingredients; anticancer ingredients, including antineoplastic drugs; anticholinergics; anticonvulsants; antidepressants; antidiabetic ingredients; antidiarrheals; antihelminthics; antihistamines; antihyperlipidemic ingredients; antihypertensive ingredients; anti-infective ingredients such as antibiotics and antiviral ingredients; antiinflammatory ingredients; antimigraine preparations; antinauseants; antiparkinsonism drugs; antipruritics; antipsychotics; antipyretics; antispasmodics; antitubercular ingredients; antiulcer ingredients; anxiolytics; appetite suppressants; attention deficit disorder (ADD) and attention deficit hyperactivity disorder (ADHD) drugs; cardiovascular preparations including calcium channel blockers, CNS ingredients; beta-blockers and antiarrhythmic ingredients; central nervous system stimulants; cough and cold preparations, including decongestants; diuretics; genetic materials; herbal remedies; hormonolytics; hypnotics; hypoglycemic ingredients; immunosuppressive ingredients; leukotriene inhibitors; mitotic inhibitors; muscle relaxants; narcotic antagonists; nicotine; nutritional ingredients, such as vitamins, essential amino acids and fatty acids; ophthalmic drugs such as antiglaucoma ingredients; parasympatholytics; psychostimulants; sedatives; steroids; sympathomimetics; tranquilizers; and vasodilators including general coronary, peripheral and cerebral.

The pharmaceutical ingredient may also be a biomolecule, e.g., a molecular moiety selected from the group consisting of DNA, RNA, antisense oligonucleotides, peptidyl drugs, i.e., peptides, polypeptides and proteins (including fluorescent proteins), ribosomes and enzyme cofactors such as biotin. Biomolecules (as well as other ingredients) may be radioactively tagged or otherwise labeled for diagnostic purposes, as will be discussed in further detail below.

Suitable pharmacologically active peptides will generally although not necessarily have a molecular weight of at least 300 Da, and preferably at least 800 Da. Examples of such peptides which may be substantially stable in the extended release formulations over the intended period of release, and which may therefore be used in the compositions of this invention, are oxytocin, vasopressin, adrenocorticotropic hormone (ACTH), epidermal growth factor (EGF), prolactin, luteinizing hormone, follicle stimulating hormone, luliberin or luteinizing hormone releasing hormone (LHRH), insulin, somatostatin, glucagon, interferon, gastrin, tetragastrin, pentagastrin, urogastrone, secretin, calcitonin, enkephalins, endorphins, kyotorphin, taftsin, thymopoietin, thymosin, thymostimulin, thymic humoral factor, serum thymic factor, tumour necrosis factor, colony stimulating factors, motilin, bombesin, dinorphin, neurotensin, cerulein, bradykinin, urokinase, kallikrein, substance P analogues and antagonists, angiotensin II, nerve growth factor, blood coagulation factors VII and IX, lysozyme chloride, renin, bradykinin, tyrocidin, gramicidines, growth hormones, melanocyte stimulating hormone, thyroid hormone releasing hormone, thyroid stimulating hormone, parathyroid hormone, pancreozymin, cholecystokinin, human placental.lactogen, human chorionic gonadotropin, protein synthesis stimulating peptide, gastric inhibitory peptide, vasoactive intestinal peptide, platelet derived growth factor, growth hormone releasing factor, bone morphogenic protein, and synthetic analogues and modifications and pharmacologically active fragments thereof. Peptidyl drugs also include synthetic analogs of LHRH, e.g., buserelin, deslorelin, fertirelin, goserelin, histrelin, leuprolide (leuprorelin), lutrelin, nafarelin, tryptorelin, and pharmacologically active salts thereof.

Any suitable salts of active pharmaceutical ingredients may be used including, for example, sodium, hydrochloride, potassium, mesylate, axetil, phosphate, succinate, maleate. Alternatively, the free acid form of active agents may be used.

In another embodiment, the invention includes a process for preparing combination products containing two or more substances using supercritical fluid technology.

The substances of interest to be prepared in the combination product may be any molecular entity. Those substances that are particularly suited to uses involving particles are preferred. The uses for such combination products include cosmetics, foodstuffs, polymer technology (including plastics, fibers, biopolymers, etc.), chemical reagents, catalysts, energy storage materials, fuel cells, propellants, ceramics, microelectronics, photographic film and developer products, colorants (including pigments, dyes, etc.), phosphors, powder metallurgy products, ceramics, papermaking technology, etc.

The following examples of substances useful in preparing combination products of interest according to the invention and uses thereof. These examples are for purposes of illustration and are not intended to be limiting.

Catalysts: Generally although not necessarily metal-based, comprised of a single metal, a mixture or alloy of two or more metals, or an organometallic complex (e.g., metallocenes, Ziegler-Natta catalysts).

Ceramics: Generally although not necessarily based on oxides, carbides, nitrides, borides, and silicates, including, for example, silicon nitride, silicon oxynitride, silicon carbide, tungsten carbide, tungsten oxycarbide, molybdenum carbide, aluminum oxide, calcium oxide, magnesium oxide, titanium oxide, aluminum silicates (e.g., sillimanite and mullite), magnesium silicates (forsterite), zirconium silicates (zircon), magnesium aluminum oxide (spinel), etc.

Metals: Industrially or otherwise useful metal particles may be comprised of any metal or metallic alloy or composite, e.g., silver, gold, copper, lithium, aluminum, platinum, palladium, or the like.

Semiconductor materials include, but are not limited to, silicon, silicon dioxide, other metal oxides, germanium, and silicon-germanium. Semiconductors also include those comprised of a first element selected from Group 13 of the Periodic Table of the Elements and a second element selected from Group 15 (GaN, GaP, GaAs, GaSb, InN, InP, InAs, InSb, and the like); and those comprised of a first element selected from Groups 2 and 12 of the Periodic Table of the Elements and a second element selected from Group 16 (e.g., ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like).

Conductive and semiconductive organics are typically conjugated polymers, for example, cis and trans polyacetylenes, polydiacetylenes, polyparaphenylenes, polypyrroles, polythiophenes, polybithiophenes, polyisothianaphthene, polythienylvinylenes, polyphenylenesulfide, polyaniline, polyphenylenevinylenes, and polyphenylenevinylene derivatives, e.g., poly(2-methoxy-5-(2-ethylhexyloxy)-1,4-phenylene vinylene (“MEH-PPV”) (see U.S. Pat. No. 5,189,136 to Wudl et al.), poly(2,5-bischelostanoxy-1,4-phenylene vinylene) (“BCHA-PPV”) (e.g., as described in International Patent Publication No. WO 98/27136), and poly(2-N,N-dimethylamino phenylene vinylene)(described in U.S. Pat. No. 5,604,292 to Stenger-Smith et al.).

Capacitor materials: Particles useful in capacitors include polyester, polypropylene, polystyrene, glass, silica, mica, silver mica, aluminum oxide, tantalum oxide, and barium titanate.

Colorants include dyes and pigments. Dyes include azo or “direct” dyes as well as disperse dyes and dyes containing reactive groups, e.g., dyes containing acidic groups (e.g., carboxylate, phosphonate or sulfonate moieties), basic groups (e.g., unsubstituted amines or amines substituted with 1 or 2 alkyl, typically lower alkyl, groups), or both. Dyes may also be luminescent, e.g., from the fluorescein, rhodamine, pyrene and porphyrin families. Inorganic pigments include, for example, iron blue, titanium dioxide, red iron oxide, strontium chromate, hydrated aluminum oxide, zinc oxide, zinc sulfide, lithopone, antimony oxide, zirconium oxide, kaolin (hydrous aluminosilicate), and carbon black.

Organic pigments include, without limitation: azo pigments such as azo lake pigments, insoluble azo pigments, condensed azo pigments, and chelated azo pigments; polycyclic pigments such as phthalocyanine pigments, perylene pigments, perynone pigments, anthraquinone pigments, quinacridone pigments, dioxazine pigments, thio-indigo pigments, isoindolinone pigments, and quinophthalone pigments; nitro pigments; nitroso pigments; and aniline black.

Energy storage materials: In high voltage systems, examples of suitable particles for use in anodes include, but are not limited to, lithium, lithium/aluminum alloys, carbon, graphite, nitrides, and tin oxide. Suitable particles for use in cathodes include manganese oxide (spinel), lithium cobalt oxide, lithium nickel oxide, vanadium oxide, iron oxide, mixed metal oxides, iron sulfide, copper sulfide, CFx, iodine, sulfur, mixed metal sulfides, metal and mixed metal phosphates.

Battery Applications: Particles for use as anodes in alkaline battery applications include, but are not limited to, zinc and various zinc alloys with, e.g., lead, mercury, indium, tin, etc. Suitable alkaline cathodes include, for example, manganese dioxide, silver oxide with graphite and carbon for electronic conduction. Metal hydride battery electrode materials are typically nickel alloys with lanthanum and other trace elements.

Fuel cells: In direct methanol fuel cells platinum-ruthenium alloy particles or particles made from platinum-based alloys in which a second metal is tin, iridium, osmium, or rhenium are suitable for use as anodes. Cathodes may be prepared from platinum particles.

Photographic applications: Examples of particles that may be used in photographic applications include, but are not limited to, silver halides such as silver chloride, silver bromide, silver bromoiodide, and dye sensitive variants thereof.

Phosphors: Phosphors are normally composed of inorganic luminescent materials that absorb incident radiation and subsequently emit radiation within the visible region of the spectrum. Phosphors are preferably capable of maintaining luminescence (e.g., fluorescence) under excitation for a relatively long period of time to provide superior image reproduction. Various phosphors include, for example, Y₂ O₃:Eu, ZnS:Ag, Zn₂SiO₄:Mn, ZnO:Zn, and other doped rare earth metal oxides.

Powder metallurgy products: Examples of suitable powder metallurgy particles include tungsten copper, silver tungsten, silver graphite, silver nickel, tungsten molybdenum, high density tungsten based heavy metals, tungsten carbide. Other ferrous and non-ferrous particles include iron and steel, iron, copper steel, iron nickel steel, low alloy steels, sinter hardened steels, and copper infiltrated steels, along with a variety of bronze, copper and brass materials.

Resins: Examples of synthetic resin particles include, without limitation, polyester resin particles, polyamide resin particles, polyvinyl chloride resin particles, polyurethane resin particles, urea resin particles, polystyrene resin particles, particles of styrene-acrylic copolymers (copolymers of styrene and derivatives of (meth)acrylic acid), polymethyl methacrylate particles, melamine resin particles, epoxy resin particles, and silicone resin particles. A wide variety of other polymeric particles are also useful, e.g., in plastics technology, fiber manufacturing, etc.

Conventional processes using supercritical fluids for producing pharmaceutical particles may be used. Examples of preferred supercritical processing techniques for recrystallizing pharmaceuticals include Rapid Expansion from Supercritical Solutions (RESS), Supercritical Anti-Solvent (SAS), and Gas Antisolvent (GAS). Another example of a suitable process for use in the present invention is the Supercritical Antisolvent Precipitation with Enhanced Mass Transfer (SAS-EM) technique.

Further techniques suitable for preparing combination pharmaceutical formulations include U.S. Pat. No. 6,620,351, U.S. Pat. No. 5,707,634, U.S. Pat. No. 5,360,478, U.S. Pat. No. 5,043,280, U.S. Pat. No. 4,582,731, European Patent EP 0 542 314 and Larson, K. A., Biotechnol. Prog., 2:73-82 (1986).

These supercritical fluid processes involve the use of a solvent, also referred to as a solvent, in preparing the pharmaceutical combinations. Examples of solvents useful in the processes of the present invention include, without limitation, the following: water, hydrocarbons, including aliphatic alkanes such as hexane, heptane, decalin, octane, etc., cyclic alkanes such as cyclohexane, and aromatic hydrocarbons such as benzene, cumene, pyridine, pseudocumene, cymene, styrene, toluene, xylenes, tetrahydronaphthalene and mesitylene; halogenated compounds such as carbon tetrachloride and chlorinated, fluorinated and brominated hydrocarbons such as chloroform, bromoform, methyl chloroform, chlorobenzene, o-dichlorobenzene, chloroethane, 1,1-dichloroethane, 1,2-dichloroethane, tetrachloroethane, epichlorohydrin, trichloroethylene and tetrachloroethylene; ethers such as diethyl ether, diisopropyl ether, diisobutyl ether, diglyme, 1,4-dioxane, 1,3-dioxolane, dimethoxymethane, furan and tetrahydrofuran; aldehydes such as methyl formate, ethyl formate and furfural; ketones such as acetone, diisobutyl ketone, cyclohexanone, methyl ethyl ketone, N-methyl-2-pyrrolidone and isophorone; amides such as dimethyl formamide and dimethyl acetamide; alcohols such as ethanol, isopropanol, n-propanol, t-butyl alcohol, cyclohexanol, 1-hexanol, 1-octanol and trifluoroethanol; polyhydric alcohols such as 1,3-propanediol, glycerol, ethylene glycol, propylene glycol, and low molecular weight (typically less than 400) polyethylene glycol; amines, including cyclic amines such as pyridine, piperidine, 2-methylpyridine, morpholine, etc., and mono-, di- and tri-substituted amines such as trimethylamine, dimethylamine, methylamine, triethylamine, diethylamine, ethylamine, n-butylamine, t-butylamine, triethanolamine, diethanolamine and ethanolamine, and amine-substituted hydrocarbons such as ethylene diamine, diethylene triamine; carboxylic acids such as acetic acid, trifluoroacetic acid and formic acid; esters such as ethyl acetate, isopentyl acetate, propylacetate, etc.; lactams such as caprolactam; nitriles such as acetonitrile, propane nitrile and adiponitrile; organic nitrates such as nitrobenzene, nitroethane and nitromethane; and sulfides such as carbon disulfide.

The solvent may optionally be a lipidic material including, but not limited to, the following: phospholipids such as phosphorylated diacyl glycerides, and particularly phospholipids selected from the group consisting of diacyl phosphatidylcholines, diacyl phosphatidylethanolamines, diacyl phosphatidylserines, diacyl phosphatidylinositols, diacyl phosphatidylglycerols, diacyl phosphatidic acids, and mixtures thereof, wherein each acyl group contains about 10 to about 22 carbon atoms and is saturated or unsaturated; fatty acids such as isovaleric acid, valeric acid, caproic acid, enanthic acid, caprylic acid, pelargonic acid, capric acid, lauric acid, myristic acid, palmitic acid, stearic acid, arachidic acid, behenic acid, lignoceric acid, oleic acid, linoleic acid, linolenic acid, and arachidonic acid; lower fatty acid esters comprising esters of the foregoing fatty acids, wherein the carboxylic acid group of the fatty acid is replaced with an ester moiety —(CO)—OR wherein R is a C₁₋₃ alkyl moiety optionally substituted with one or two hydroxyl groups; fatty alcohols corresponding to the aforementioned fatty acids, wherein the carboxylic acid group of the fatty acid is replaced by a —CH₂OH group; glycolipids such as cerebroside and gangliosides; oils, including animal oils such as cod liver oil and, menhaden oil, and vegetable oils such as babassu oil, castor oil, corn oil, cotton seed oil, linseed oil, mustard oil, olive oil, palm oil, palm kernel oil, peanut oil, poppyseed oil, rapeseed oil, safflower oil, sesame oil, soybean oil, sunflower seed oil, tung oil or wheat germ oil; and waxes, i.e., higher fatty acid esters, including animal waxes such as beeswax and shellac, mineral waxes such as montan, petroleum waxes such as microcrystalline wax and paraffin, and vegetable waxes such as carnauba wax.

In the RESS process, the two or more active pharmaceutical ingredients are dissolved in a supercritical fluid, preferably carbon dioxide, to form a homogenous solution. Other excipients may optionally be added to the supercritical fluid. The active agents and optional excipients may be added to the supercritical fluid simultaneously or other suitable order. The resulting solution is then passed through an orifice or nozzle into a chamber. Preferably, the pressure in the chamber is atmospheric. By spraying the homogenous solution through an orifice or nozzle, the solution is depressurized rapidly resulting in the vaporization of the carbon dioxide or other supercritical fluid. The active agents and optional excipients are recrystallized as a uniform mixture in dry powder form.

RESS can be used if the active pharmaceutical ingredients to be precipitated are soluble in the supercritical fluid, such as supercritical carbon dioxide. If the active agents are not readily soluble in the supercritical fluid, the active agents may be first dissolved in a cosolvent system and then added to the supercritical fluid. The cosolvent may be a single solvent or two or more solvents combined together. Alternatively, the cosolvent may be added to the supercritical fluid initially followed by the addition of the active agents to the mixture of the supercritical fluid and cosolvent. When a cosolvent is required, the cosolvent used generally has a higher dielectric constant than the supercritical fluid (e.g., supercritical carbon dioxide), but is miscible in the supercritical fluid.

Examples of suitable solvents and cosolvents include acetone, methanol, ethanol, propanol, butanol, tetrahydrorfuran, methylene chloride, chloroform, toluene, dimethylsufloxide, N,N-dimethylfornamide, cyclohexanone, butrylactone, water, and combinations thereof. Other suitable solvents include those compounds known in the art in which the active pharmaceutical ingredients to be blended can be dissolved.

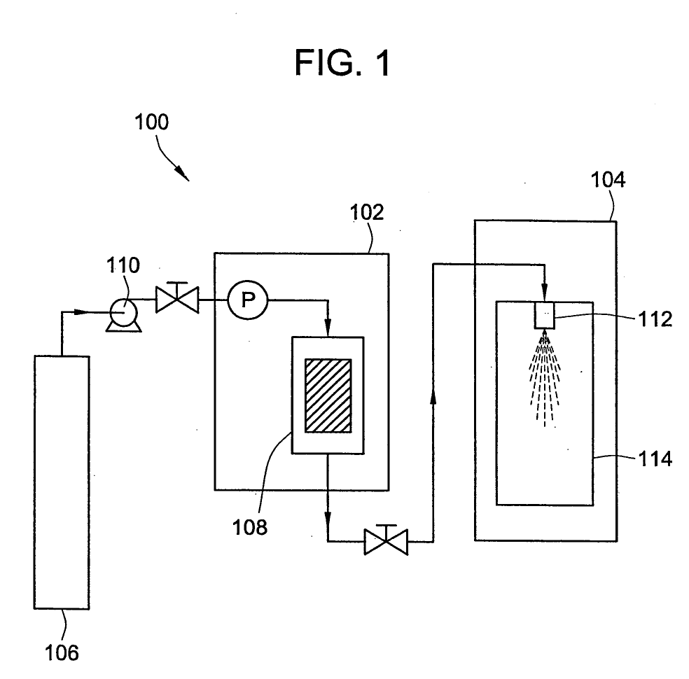

A typical flow diagram of a RESS process for recrystallization using carbon dioxide as the supercritical fluid is shown in FIG. 1. The RESS apparatus 100 generally includes an extraction unit 102 and precipitation unit 104. Carbon dioxide is transferred from storage tank 106 to high-pressure vessel 108, optionally using pump 110. The temperature and pressure in high-pressure vessel 108 are maintained such that the carbon dioxide exists in a supercritical state. The active pharmaceutical ingredients are then added to high-pressure vessel 108 to form a homogenous solution of the carbon dioxide in which the active agents are dissolved. Alternatively, the active pharmaceutical ingredients may be added to the high-pressure vessel 108 initially followed by the addition of supercritical carbon dioxide to form a homogeneous solution. The homogenous solution is sprayed through nozzle 112 into vessel 114, preferably under atmospheric pressure conditions. Alternatively, pressures greater than atmospheric pressure may be used. The supercritical carbon dioxide is vaporized and the active agents precipitate from the solution in the form of a dry powder. The carbon dioxide may either be collected for possible reuse or discarded. The solid precipitate is collected from vessel 114 for further processing.

The upstream and downstream temperatures and pressures in the RESS process may be modified to obtain the desired morphology of the precipitated drug product. In addition, the shape of the nozzle employed may be altered to transition between fibers and particles. A smaller length-to-nozzle diameter ratio (L/D) typically results in the formation of particles.

Another suitable process for recrystallization according to the present invention is the SAS process. The SAS technique is well-suited for precipitation of active agents that are only slightly soluble in the supercritical fluid of interest, such as supercritical carbon dioxide.

In the SAS process, the active pharmaceutical ingredients and optional excipients are dissolved in a solvent. The solvent may be any suitable liquid containing one or more solvents in which the active agents are dissolved. The solvent is also miscible in the supercritical fluid. Examples of solvents suitable for use in the SAS method include those solvents discussed herein that may be used in the RESS process as cosolvents as well as other solvents in which the active agents can be dissolved.

The solution containing the active agents is then contacted with a supercritical fluid (e.g., supercritical carbon dioxide). Preferably, mixing is carried out by spraying the solution through a nozzle into a chamber filled with the supercritical fluid. The supercritical fluid acts as an anti-solvent to extract out the cosolvent. The active agents and optional excipients form a precipitate upon contact with the supercritical fluid which is recovered. The precipitate from the SAS process is a uniformly mixed dry powder containing the active pharmaceutical ingredients and any optional excipients.

The supercritical fluid may optionally be contacted one or more cosolvents prior to the addition of the solution containing the active agents.

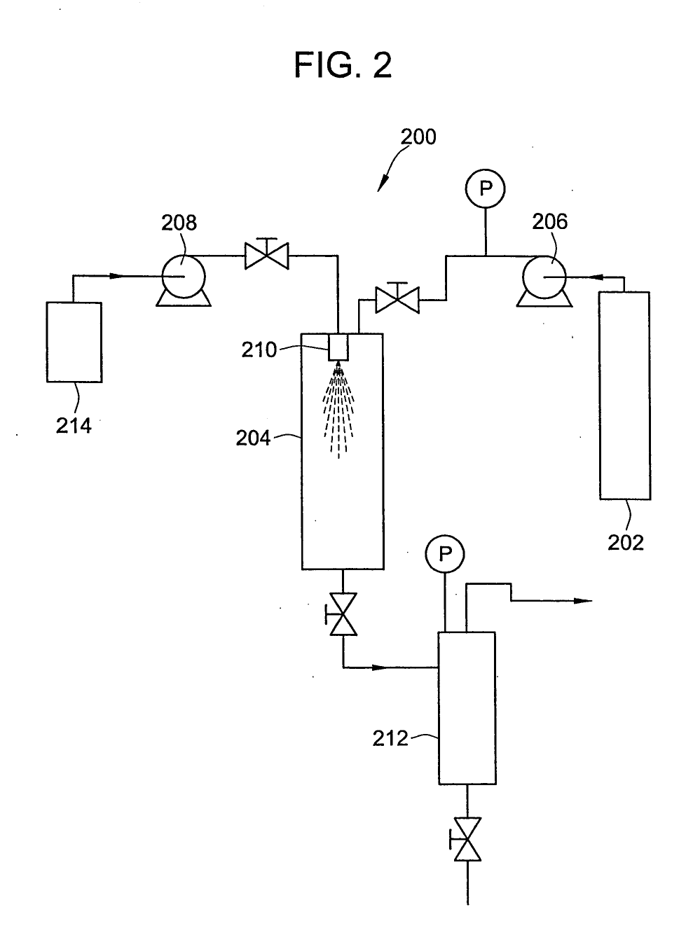

A typical flow diagram of a SAS process for recrystallization using supercritical carbon dioxide is shown in FIG. 2. In the SAS apparatus 200, the active pharmaceutical ingredients are dissolved in a solvent system in vessel 214. Excipients may optionally be dissolved in a solvent along with the active agents. Carbon dioxide is transferred from vessel 202 to high-pressure vessel 204, optionally using pump 206, wherein carbon dioxide is maintained in a supercritical state. The solvent solution containing the active agents is transferred from vessel 214, optionally using pump 208, and sprayed through nozzle 210 into high-pressure vessel 204. The precipitate containing a powder blend of active agents is recovered from high-pressure vessel 204 for fuirther processing. The resulting mixture of the solvents and supercritical carbon dioxide is then transferred to low-pressure tank 212 for recovery of the solvent and carbon dioxide and reuse of these process streams.

In the GAS process, supercritical carbon dioxide is added to a solution of the desired active pharmaceutical ingredients dissolved in an organic cosolvent. The supercritical carbon dioxide and organic solvent are miscible whereas the solid active agents have limited solubility in carbon dioxide. Thus, the carbon dioxide acts as an antisolvent to precipitate solid crystals of the active agents.

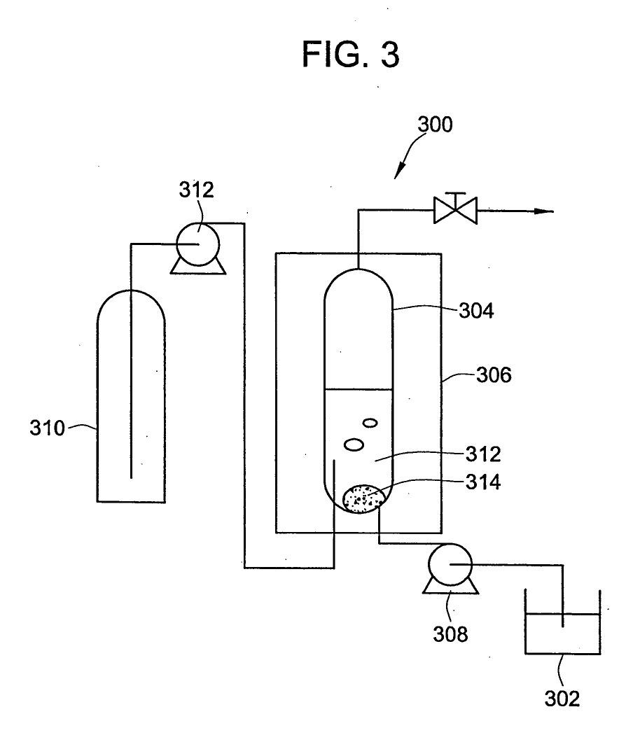

A typical flow diagram of a GAS process for recrystallization using supercritical carbon dioxide is shown in FIG. 3. In the GAS apparatus 300, the active pharmaceutical ingredients are dissolved in a solvent in vessel 302. Excipients may optionally be dissolved in the solvent along with the active agents. The solution in which the active agents are dissolved is transferred to a vessel 304 in the precipitator 306 using pump 308. Carbon dioxide stored in a supercritical state in vessel 310 is rapidly transferred to vessel 304 using pump 312. Alternatively, carbon dioxide may be stored as either a gas or liquid well below its critical temperature and critical pressure and then rendered supercritical before combining the carbon dioxide with the dissolved active agents. Upon contact with the supercritical carbon dioxide, the dissolved active agents in the solution 312 in vessel 304 are crystallized as particles 314 containing a blend of the active agents and optional excipients. The particles are recovered for further processing to yield a suitable pharmaceutical formulation.

The supercritical fluid may optionally be contacted with one or more solvents prior to the addition of the solution containing the active agents.

Other suitable processes known to those persons of ordinary skill in the art that involve a supercritical fluid, preferably supercritical carbon dioxide, may be used to recrystallize combinations of active pharmaceutical ingredients in a dry powder form.

The uniform blend of active pharmaceutical agents recrystallized using a supercritical fluid according to the process of the present invention is a powder, also referred-to herein as a dry powder. The precipitated powder typically contains about 10% or less (by weight) of the solvent in which the active agents are dissolved prior to crystallization. Preferably, the dry powder contains 5% or less solvent (by weight) and, most preferably, 2% or less (by weight) solvent.

The pharmaceutical formulations produced according to the present invention may optionally contain pharmaceutically acceptable excipients such as, for example, carriers, additives, and diluents. Pharmaceutical formulations for parenteral administration may contain, for example, alkylene glycols such as propylene glycol, polyalkylene glycols such as polyethylene glycol, oils of vegetable origin, hydrogenated naphthalenes, acid or basic buffers, and the like.

Other examples of suitable excipients for pharmaceutical dosage forms prepared by the present invention include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. Pharmaceutical formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The pharmaceutical active ingredients used in the present invention may be premixed with one or more pharmaceutically acceptable excipients before the active agents are contacted with a supercritical fluid according to the inventive processes. When premixed with the active agents, the excipients must be compatible with the cosolvent systems and supercritical fluids that are employed.

Alternatively, a uniform blend of two or more active pharmaceutical ingredients obtained by recrystallization using a supercritical fluid may be contacted with one or more pharmaceutically acceptable excipients to produce a pharmaceutical formulation.

The following examples further illustrate the invention but, of course, should not be construed as in any way limiting its scope.

### EXAMPLES

The examples demonstrate the process for producing pharmaceutical formulations using a supercritical fluid according to the present invention. Examples 1-11 describe pharmaceutical formulations containing sodium sulbactam and sodium ampicillin. Examples 12-21 describe pharmaceutical formulations containing etoposide and paclitaxel. The samples of Examples 1-21 are prepared by one of the following three processes.

Process 1

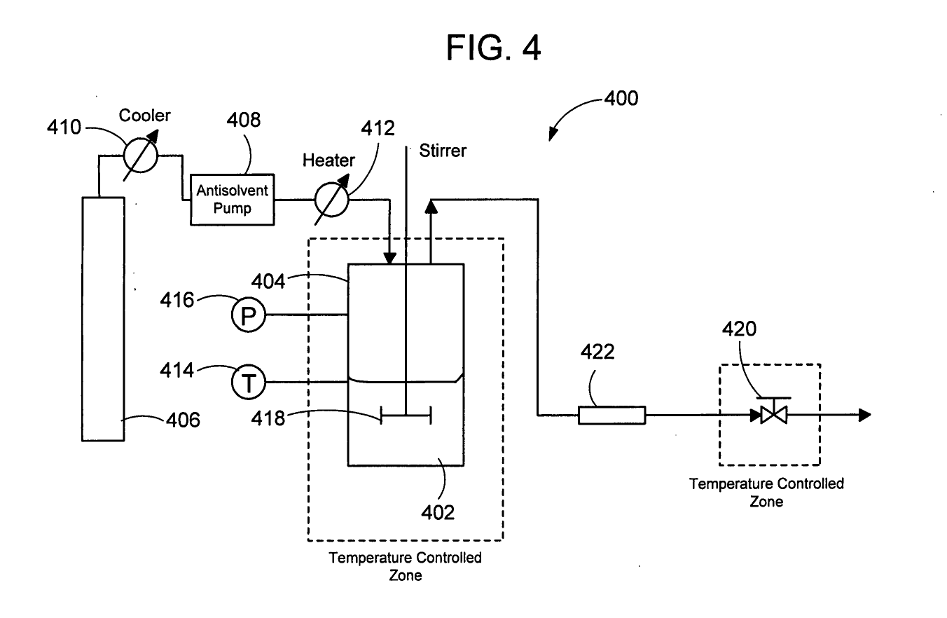

The flow diagram for process 1 is shown in FIG. 4. In apparatus 400, the active pharmaceutical ingredients are dissolved in a cosolvent to form a drug solution 402 in vessel 404. After closing vessel 404, antisolvent from vessel 406 is slowly added to the drug solution 402 in vessel 404. The antisolvent may optionally be pumped to vessel 404 using pump 408. The temperature of the antisolvent may optionally be adjusted with cooler 410 and/or heater 412. Temperature is steadily maintained during the antisolvent addition to the drug solution 402, for example by thermocouple 414. Due to the antisolvent addition, pressure in vessel 404 rises steadily. The antisolvent addition rate may also be monitored by recording the rate of increase of pressure in vessel 402 using pressure monitor 416. Antisolvent diffuses into the drug solution 402 while the cosolvent diffuses into the antisolvent, resulting in the precipitation of the active agents as a powder. A magnetic stirrer 418 is employed to obtain uniform mixing in vessel 404. After the pressure reaches a desired value, a pressure control valve 420 in the outlet line is opened to control the pressure in vessel 402. The antisolvent may optionally be filtered through filter 422. Antisolvent flow rate from vessel 404 is maintained constant for a period of time to remove any residual cosolvent present in the precipitated powder.

Preferably, the temperature of vessel 404 is maintained at a temperature appropriate to avoid substance degradation, if any. Additional stabilizing agents may be added to maintain pH of the solution in case of aqueous solutions. Further substances may be added to protect any substance degradation caused during the solution preparation or processing.

Process 2

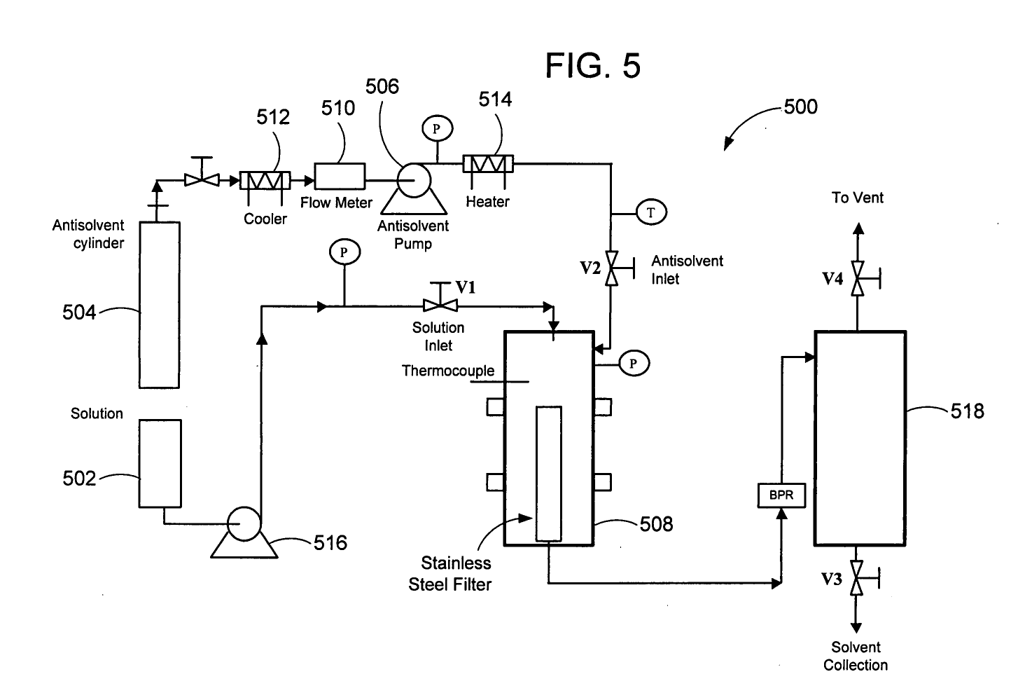

The flow diagram for process 2 is shown in FIG. 5. In apparatus 500, the active pharmaceutical ingredients are dissolved in a cosolvent in the solution feed vessel 502 and stirred, making a uniform solution. A flow of antisolvent contained in vessel 504 (e.g., CO₂ cylinder) is maintained at a desired temperature and pressure using antisolvent pump 506 to the particle production vessel 508. The antisolvent flow is monitored with flow meter 510. Optionally, the antisolvent may be cooled by heat exchanger 512 or heated by heat exchanger 514 prior to contact with the drug solution. The solution is dispersed using solution pump 516 into vessel 508 as a fine stream through a capillary nozzle. The antisolvent effect precipitates the substance combination as a powder. Powder is then filtered and the antisolvent/cosolvent mixture is allowed to exit vessel 508. The antisolvent/cosolvent mixture is further separated in solvent collection vessel 518 and may be recycled. The particle production vessel 508 may optionally be jacketed to permit contact with a cooling or heating coil to maintain the desired temperature in vessel 508.

Process 3

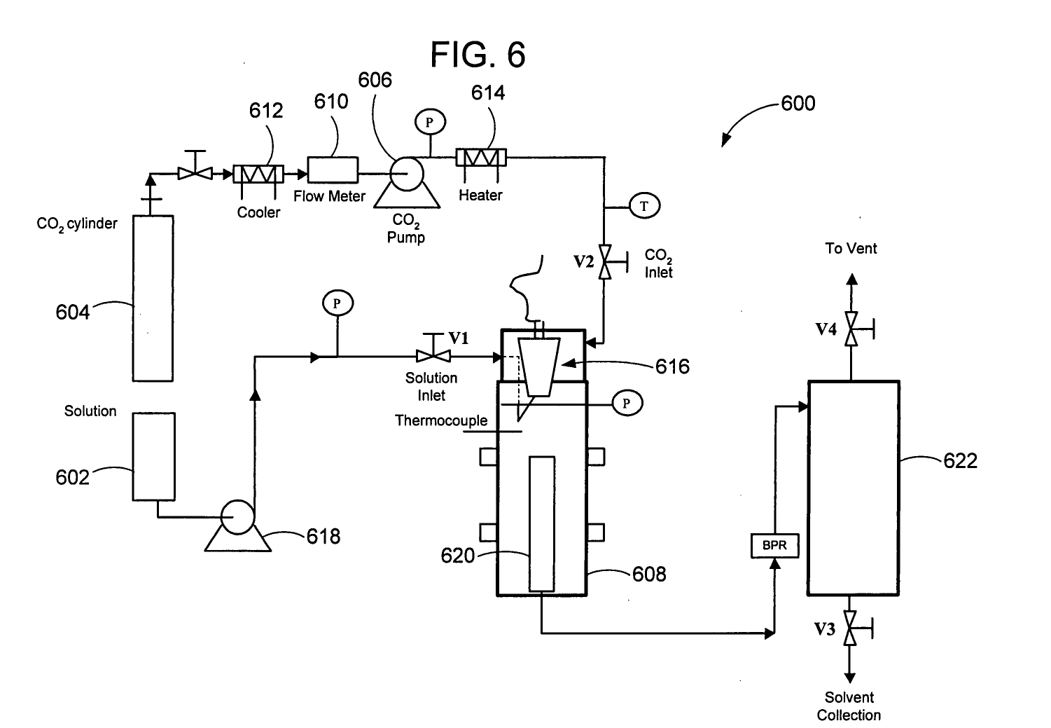

The flow diagram for process 3 is shown in FIG. 6. In apparatus 600, the active pharmaceutical ingredients are dissolved in a cosolvent in the solution feed vessel 602 and stirred making it a uniform solution. Antisolvent is contained in vessel 604 which may optionally be a CO₂ cylinder. The flow of antisolvent is maintained at a desired temperature and pressure from vessel 604 using pump 606 into the particle production vessel 608. The antisolvent flow is monitored with flow meter 610. Optionally, the antisolvent may be cooled by heat exchanger 612 or heated by heat exchanger 614 prior to contact with the drug solution. A solid surface 616 is vibrated at a desired frequency. The drug solution from vessel 602 is applied using solution pump 618 onto the vibrating surface 616 which results in uniform atomization. Antisolvent effect precipitates the substance combination as a powder. The powder is filtered using a stainless steel filter 620 and the antisolvent/solvent mixture is allowed to exit vessel 608 into the solvent collection vessel 622 and may be recycled. The particle production vessel 608 may optionally be jacketed to permit contact with a cooling or heating coil to maintain the temperature in vessel 608.

Examples 1-11 demonstrate the process for producing pharmaceutical formulations containing sodium sulbactam and sodium ampicillin using a supercritical fluid according to the present invention. For each of Examples 1-11, the ratio of sodium sulbactam to sodium ampicillin used was 1:2.

### Example 1

The desired substance combination of sodium sulbactam and sodium ampicillin was made into an aqueous solution and processed using process 1. A compressed carbon dioxide/ethanol mixture was used as the antisolvent. The concentrations of sodium sulbactam and sodium ampicillin in the aqueous solution were 200 mg/ml and 400 mg/ml, respectively. About 15 mL of the solution was dispensed in the vessel. The temperature of the precipitation vessel was maintained at about 35° C. A 20 ml high pressure vessel was used as vessel 402 in FIG. 4, and pressure of the system was maintained at 100 bar. The antisolvent flow consisted of 12 g/min of carbon dioxide and 1.5 ml/min (at atmospheric conditions) of ethanol. After the pressure reached 100 bar, antisolvent flow was maintained for 60 minutes. At the end of 60 minutes, the ethanol flow was stopped and carbon dioxide flow was maintained for an additional 15 minutes. The vessel was opened and the powder material was collected, weighed, labeled and stored.

### Example 2

The desired substance combination of sodium sulbactam and sodium ampicillin was made into an aqueous solution and processed using process 1. Compressed carbon dioxide/ethanol mixture was used as the antisolvent. The concentration of sodium sulbactam and sodium ampicillin in the aqueous solution were 200 mg/ml and 400 mg/ml, respectively. About 15 ml of the solution was dispensed in the vessel. The temperature of the precipitation vessel was maintained at about 60° C. A 20 ml high pressure vessel was used as vessel 402 in FIG. 4, and pressure of the system was maintained at 100 bar. The antisolvent flow consisted of 12 g/min of carbon dioxide and 1.5 ml/min (at atmospheric conditions) of ethanol. After the pressure reached 100 bar, antisolvent flow was maintained for 60 minutes. At the end of 60 minutes, the ethanol flow was stopped and carbon dioxide flow was maintained for an additional 15 minutes. The vessel was opened and the powder material was collected, weighed, labeled and stored.

### Example 3

Sodium sulbactam and sodium ampicillin were dissolved in an acetate buffer (pH=7.0) by dissolving 92.21 mg of sodium acetate trihydrate per ml of water and 18.4 microliter of acetic acid per ml of water. 50 ml of the abovementioned solution was mixed with 450 ml of methanol and the resultant solution was processed using process 3. Compressed carbon dioxide was used as the antisolvent. The concentrations of sodium sulbactam and sodium ampicillin in the aqueous solution were 8 mg/ml of water and 16 mg/ml of water, respectively. The temperature of the precipitation vessel was maintained at about 35° C. A 500 ml high pressure vessel was used as the particle production vessel 608 in FIG. 6, and pressure of the system was maintained at 100 bar. The antisolvent flow consisted of 50 g/min of carbon dioxide. The solution flow rate was maintained at 0.5 ml/min for 150 minutes. A 200 micron capillary tube was used to apply the solution on to the vibrating surface. A Branson 900 BCA system with a frequency of 20 kHz was used at 50% amplitude to vibrate the atomizing surface. After the end of solution flow, carbon dioxide flow was maintained for 60 additional minutes. At the end of 60 minutes, the system was depressurized. The vessel was opened and the powder material was collected, weighed, labeled and stored.

### Example 4

Sodium sulbactam and sodium ampicillin were dissolved in distilled water. The resultant solution was processed using process 3. Compressed carbon dioxide/ethanol mixture was used as the antisolvent. Concentration of sodium sulbactam and sodium ampicillin in the aqueous solution were 80 mg/ml of water and 160 mg/ml of water respectively. The temperature of the precipitation vessel was maintained at about 35° C. A 500 ml high pressure vessel was used as the particle production vessel 608 in FIG. 6. Pressure of the system was maintained at 100 bar. The antisolvent flow consisted of 50 g/min of carbon dioxide and 6 ml/min of ethanol. The solution flow rate was maintained at 0.5 ml/min for 45 minutes. A 200 micron capillary tube was used to apply the solution on to the vibrating surface. A Branson 900 BCA system with a frequency of 20 kHz was used at 50% amplitude to vibrate the atomizing surface. After the end of solution flow, carbon dioxide/ethanol flow was maintained for 60 additional minutes. At the end of 60 minutes, ethanol flow was stopped and carbon dioxide flow was continued for additional 30 minutes and the system was depressurized. The vessel was opened and the powder material was collected, weighed, labeled and stored.

### Example 5

Sodium sulbactam and sodium ampicillin was dissolved in an acetate buffer (pH=7.0) by dissolving 92.21 mg of sodium acetate trihydrate per ml of water and 18.4 microliter of acetic acid per ml of water. 50 ml of the above mentioned solution is mixed with 450 ml of methanol and the resultant solution was processed using process 3. Compressed carbon dioxide was used as the antisolvent. The concentration of sodium sulbactam and sodium ampicillin in the aqueous solution were 8 mg/ml of water and 16 mg/ml of water respectively. The temperature of the precipitation vessel was maintained at about 35° C. A 500 ml high pressure vessel was used as the particle production vessel 608 in FIG. 6. Pressure of the system was maintained at 100 bar. The antisolvent flow consisted of 50 g/min of carbon dioxide. The solution flow rate was maintained at 0.5 ml/min for 150 minutes. A 200 micron capillary tube was used to apply the solution on to the vibrating surface. A Branson 900 BCA system with a frequency of 20 kHz was used at 50% amplitude to vibrate the atomizing surface. After the end of solution flow, carbon dioxide flow was maintained for 90 additional minutes. At the end of 60 minutes, the system was depressurized. The vessel was opened and the powder material was collected for further processing.

### Example 6

Sodium sulbactam and sodium ampicillin was dissolved in distilled water. The resultant solution was processed using process 3. Compressed carbon dioxide/ethanol mixture was used as the antisolvent. Concentration of sodium sulbactam and sodium ampicillin in the aqueous solution were 160 mg/ml of water and 320 mg/ml of water respectively. The temperature of the precipitation vessel was maintained at about 35° C. A 500 ml high pressure vessel was used as the particle production vessel 608 in FIG. 6. Pressure of the system was maintained at 100 bar. The antisolvent flow consisted of 50 g/min of carbon dioxide and 6 ml/min of ethanol. The solution flow rate was maintained at 0.5 ml/min for 60 minutes. A 200 micron capillary tube was used to apply the solution on to the vibrating surface. A Branson 900 BCA system with a frequency of 20 kHz was used at 50% amplitude to vibrate the atomizing surface. After the end of solution flow, carbon dioxide/ethanol flow was maintained for 60 additional minutes. At the end of 60 minutes, ethanol flow was stopped; carbon dioxide flow was continued for additional 30 minutes; and the system was depressurized. The vessel was opened and the powder material was collected for fturther processing.

### Example 7

Sodium sulbactam and sodium Ampicillin was dissolved in an acetate buffer (pH=7.0) by dissolving 92.21 mg of sodium acetate trihydrate per ml of water and 18.4 microliter of acetic acid per ml of water. 50 ml of the above mentioned solution is mixed with 450 ml of methanol and the resultant solution was processed using process 3. Compressed carbon dioxide was used as the antisolvent. The concentrations of sodium sulbactam and sodium ampicillin in the aqueous solution were 8 mg/ml of water and 16 mg/ml of water, respectively. The temperature of the precipitation vessel was maintained at about 35° C. A 500 ml high pressure vessel was used as the particle production vessel 608 in FIG. 6, and pressure of the system was maintained at 100 bar. The antisolvent flow consisted of 50 g/min of carbon dioxide. The solution flow rate was maintained at 0.5 ml/min for 60 minutes. A 200 micron capillary tube was used to apply the solution onto the vibrating surface. A Branson 900 BCA system with a frequency of 20 kHz was used at 50% amplitude to vibrate the atomizing surface. After the end of solution flow, carbon dioxide flow was maintained for 60 additional minutes. At the end of 60 minutes, the system was depressurized. The vessel was opened and the powder material was collected for further processing.

### Example 8

Sodium sulbactam and sodium ampicillin were dissolved in distilled water. The resultant solution was processed using process 3. Compressed carbon dioxide/ethanol mixture was used as the antisolvent. Concentration of sodium sulbactam and sodium ampicillin in the aqueous solution were 160 mg/ml of water and 320 mg/ml of water respectively. The temperature of the precipitation vessel was maintained at about 35° C. A 500 ml high pressure vessel was used as the particle production vessel 608 in FIG. 6. Pressure of the system was maintained at 100 bar. The antisolvent flow consisted of 50 g/min of carbon dioxide and 6 ml/min of ethanol. The solution flow rate was maintained at 0.5 ml/min for 45 minutes. A 200 micron capillary tube was used to apply the solution on to the vibrating surface. A Branson 900 BCA system with a frequency of 20 kHz was used at 50% amplitude to vibrate the atomizing surface. After the end of solution flow, carbon dioxide/ethanol flow was maintained for 60 additional minutes. At the end of 60 minutes, ethanol flow was stopped and carbon dioxide flow was continued for additional 30 minutes and the system was depressurized. The vessel was opened and the powder material was collected for further processing.

### Example 9

Sodium sulbactam and sodium ampicillin was dissolved in distilled water. The resultant solution was processed using process 3. Compressed carbon dioxide/ethanol mixture was used as the antisolvent. The concentrations of sodium sulbactam and sodium ampicillin in the aqueous solution were 160 mg/ml of water and 320 mg/ml of water, respectively. The temperature of the precipitation vessel was maintained at about 35° C. A 500 ml high pressure vessel was used as the particle production vessel 608 in FIG. 6. Pressure of the system was maintained at 100 bar. The antisolvent flow consisted of 50 g/min of carbon dioxide and 6 ml/min of ethanol. The solution flow rate was maintained at 1.0 ml/min for 30 minutes. A 200 micron capillary tube was used to apply the solution onto the vibrating surface. A Branson 900 BCA system with a frequency of 20 kHz was used at 50% amplitude to vibrate the atomizing surface. After the end of solution flow, carbon dioxide/ethanol flow was maintained for 60 additional minutes. At the end of 60 minutes, ethanol flow was stopped; carbon dioxide flow was continued for additional 30 minutes; and the system was depressurized. The vessel was opened and the powder material was collected, weighed, labeled and stored.

### Example 10

Sodium sulbactam and sodium ampicillin was dissolved in distilled water in an icepack. The resultant solution was processed using process 3. Compressed carbon dioxide/ethanol mixture was used as the antisolvent. The concentrations of sodium sulbactam and sodium ampicillin in the aqueous solution were 40 mg/ml of water and 80 mg/ml of water, respectively. The temperature of the solution syringe pump was maintained at 23° C. The temperature of the precipitation vessel was maintained at about 35° C. A 500 ml high pressure vessel was used as the particle production vessel 608 in FIG. 6. Pressure of the system was maintained at 100 bar. The antisolvent flow consisted of 100 g/min of carbon dioxide and 10 ml/min of ethanol. The solution flow rate was maintained at 0.5 ml/min for 90 minutes. A 200 micron capillary tube was used to apply the solution onto the vibrating surface. A Branson 900 BCA system with a frequency of 20 kHz was used at 50% amplitude to vibrate the atomizing surface. After the end of solution flow, carbon dioxide/ethanol flow was maintained for 60 additional minutes. At the end of 60 minutes, ethanol flow was stopped; carbon dioxide flow was continued for additional 60 minutes; and the system was depressurized. The vessel was opened and the powder material was collected, weighed, labeled and stored.

### Example 11

Sodium sulbactam and sodium ampicillin was dissolved in distilled water in an icepack. The resultant solution was processed using process 3. Compressed carbon dioxide/ethanol mixture was used as the antisolvent. The concentrations of sodium sulbactam and sodium ampicillin in the aqueous solution were 40 mg/ml of water and 80 mg/ml of water, respectively. The temperature of the solution syringe pump was maintained at 23° C. The temperature of the precipitation vessel was maintained at about 35° C. A 500 ml high pressure vessel was used as the particle production vessel 608 in FIG. 6. Pressure of the system was maintained at 100 bar. The antisolvent flow consisted of 100 g/min of carbon dioxide and 10 ml/min of ethanol. The solution flow rate was maintained at 0.5 ml/min for 90 minutes. A 200 micron capillary tube was used to apply the solution on to the vibrating surface. A Branson 900 BCA system with a frequency of 20 kHz was used at 50% amplitude to vibrate the atomizing surface. After the end of solution flow, carbon dioxide/ethanol flow was maintained for 60 additional minutes. At the end of 60 minutes, ethanol flow was stopped; carbon dioxide flow was continued for additional 30 minutes; and the system was depressurized. The vessel was opened and the powder material was collected, weighed, labeled and stored.

The sample of Example 10 exhibited the most homogenous crystal structure which demonstrates blend uniformity. The chemical recovery of drug product was also maximized in Example 10, specifically about 58% recovery of sodium sulbactam and about 93% recovery of sodium ampicillin.

As demonstrated by the results of Examples 1-11, the process of the invention can be used to produce pharmaceutical formulations comprising sodium sulbactam and sodium ampicillin using a supercritical fluid in accordance with the present invention.

Examples 12-21 demonstrate the process for producing pharmaceutical formulations containing etoposide and paclitaxel using a supercritical fluid according to the present invention. The ratio of paclitaxel and etoposide used in the samples of Examples 12-21 and the cosolvent used to produce these samples are set forth in Table 2.

TABLE 2
Ratios of etoposide/paclitaxel, solvent and antisolvent

| | Etoposide/Paclitaxel | | |
|---|---|---|---|
| Example | Ratio | Solvent | Antisolvent |
| 12 | 1:1 | Methanol | Compressed CO₂ |
| 13 | 1:1 | Methanol | Compressed CO₂ |
| 14 | 1:1 | Methanol | Compressed CO₂ |
| 15 | 2:1 | Methanol | Compressed CO₂ |
| 16 | 1:1 | Methanol | Compressed CO₂ |
| 17 | 1:1 | Methanol | Compressed CO₂ |
| 18 | 1:1 | Methanol | Compressed CO₂ |
| 19 | 1:1 | Methanol | Compressed CO₂ |
| 20 | 1:1 | Methanol | Compressed CO₂ |
| 21 | 2:1 | Methanol | Compressed CO₂ |

### Example 12

Etoposide and paclitaxel were made into a solution in methanol and processed using process 1. Compressed carbon dioxide was used as the antisolvent. 0.5 g of etoposide and 0.5 g of paclitaxel were dissolved in 25 ml of methanol and dispensed in to the vessel. The temperature of the precipitation vessel was maintained at about 35° C. A 103 ml high pressure vessel was used as vessel 404 in FIG. 3. Pressure of the system was maintained at 100 bar. A magnetic stirrer at 520 rpm was used to obtain uniform mixing during prepicitation. The antisolvent flow consisted of 5 g/min of carbon dioxide. Pressure in the vessel was increased gradually at a rate of 1 bar/min. After the pressure reached 100 bar, antisolvent flow was maintained for 180 minutes. The vessel was depressurized, opened and powder material was collected, weighed, labeled and stored.

### Example 13

Etoposide and paclitaxel was made into a solution in methanol and processed using process 3. Compressed carbon dioxide was used as the antisolvent. 1.0 g of etoposide and 1.0 g of paclitaxel were dissolved in 50 ml of methanol. The temperature of the precipitation vessel was maintained at about 50° C. A 500 ml high pressure vessel was used as the particle production vessel 608 in FIG. 6. Pressure of the system was maintained at 80 bar. The antisolvent flow consisted of SOg/min of carbon dioxide. The solution flow rate was maintained at 1.0 ml/min for 58 minutes. A 100 micron capillary tube was used to apply the solution on to the vibrating surface. A Branson 900 BCA system with a frequency of 20 kHz was used at 20% amplitude to vibrate the atomizing surface. After the end of solution flow, carbon dioxide flow was maintained for 60 additional minutes and the system was depressurized. The vessel was opened and the powder material was collected, weighed, labeled and stored.

### Example 14

Etoposide and paclitaxel was made into a solution in methanol and processed using process 3. Compressed carbon dioxide was used as the antisolvent. 0.5 g of etoposide and 0.5 g of paclitaxel were dissolved in 50 ml of methanol. The temperature of the precipitation vessel was maintained at about 50° C. A 500 ml high pressure vessel was used as the particle production vessel 608 in FIG. 6. Pressure of the system was maintained at 80 bar. The antisolvent flow consisted of 50 g/min of carbon dioxide. The solution flow rate was maintained at 0.5 ml/min for 100 minutes. A 100 micron capillary tube was used to apply the solution on to the vibrating surface. A Branson 900 BCA system with a frequency of 20 kHz was used at 20% amplitude to vibrate the atomizing surface. After the end of solution flow, carbon dioxide flow was maintained for 60 additional minutes and the system was depressurized. The vessel was opened and the powder material was collected, weighed, labeled and stored.

### Example 15

Etoposide and paclitaxel was made into a solution in methanol and processed using process 3. Compressed carbon dioxide was used as the antisolvent. 1.0 g of etoposide and 0.5 g of paclitaxel were dissolved in 50 ml of methanol. The temperature of the precipitation vessel was maintained at about 50° C. A 500 ml high pressure vessel was used as the particle production vessel 608 in FIG. 6. Pressure of the system was maintained at 80 bar. The antisolvent flow consisted of 50 g/min of carbon dioxide. The solution flow rate was maintained at 1.0 ml/min for 50 minutes. A 100 micron capillary tube was used to apply the solution on to the vibrating surface. A Branson 900 BCA system with a frequency of 20 kHz was used at 20% amplitude to vibrate the atomizing surface. After the end of solution flow, carbon dioxide flow was maintained for 60 additional minutes and the system was depressurized. The vessel was opened and the powder material was collected, weighed, labeled and stored.

### Example 16

Etoposide and paclitaxel was made into a solution in methanol and processed using process 3. Compressed carbon dioxide was used as the antisolvent. 0.25 g of etoposide and 0.25 g of paclitaxel were dissolved in 50 ml of methanol. The temperature of the precipitation vessel was maintained at about 50° C. A 500 ml high pressure vessel was used as the particle production vessel 608 in FIG. 6. Pressure of the system was maintained at 80 bar. The antisolvent flow consisted of 50 g/min of carbon dioxide. The solution flow rate was maintained at 1.0 ml/min for 50 minutes. A 100 micron capillary tube was used to apply the solution on to the vibrating surface. A Branson 900 BCA system with a frequency of 20 kHz was used at 20% amplitude to vibrate the atomizing surface. After the end of solution flow, carbon dioxide flow was maintained for 60 additional minutes and the system was depressurized. The vessel was opened and the powder material was collected, weighed, labeled and stored.

### Example 17

Etoposide and paclitaxel was made into a solution in methanol and processed using process 3. Compressed carbon dioxide was used as the antisolvent. 1.0 g of etoposide and 1.0 g of paclitaxel were dissolved in 50 ml of methanol. The temperature of the precipitation vessel was maintained at about 50° C. A 500 ml high pressure vessel was used as the particle production vessel 608 in FIG. 6. Pressure of the system was maintained at 80 bar. The antisolvent flow consisted of 50 g/min of carbon dioxide. The solution flow rate was maintained at 1.0 ml/min for 50 minutes. A 100 micron capillary tube was used to apply the solution on to the vibrating surface. A Branson 900 BCA system with a frequency of 20 kHz was used at 20% amplitude to vibrate the atomizing surface. After the end of solution flow, carbon dioxide flow was maintained for 60 additional minutes and the system was depressurized. The vessel was opened and the powder material was collected, weighed, labeled and stored.

### Example 18

Etoposide and paclitaxel was made into a solution in methanol and processed using process 1. Compressed carbon dioxide was used as the antisolvent. 1.0 g of etoposide and 1.0 g of paclitaxel were dissolved in 25 ml of methanol and dispensed in to the vessel. The temperature of the precipitation vessel was maintained at about 50° C. A 103 ml high pressure vessel was used as vessel 402 in FIG. 4. Pressure of the system was maintained at 100 bar. A magnetic stirrer at 520 rpm was used to obtain uniform mixing during the precipitation. The antisolvent flow consisted of 5 g/min of carbon dioxide. Pressure in the vessel was increased gradually at a rate of 1 bar/min. After the pressure reached 100 bar, antisolvent flow was maintained for 180 minutes. The vessel was depressurized, opened and the powder material was collected, weighed, labeled and stored.

### Example 19

Etoposide and paclitaxel was made into a solution in methanol and processed using process 2. Compressed carbon dioxide was used as the antisolvent. 0.5 g of etoposide and 0.5 g of paclitaxel were dissolved in 50 ml of methanol. The temperature of the precipitation vessel was maintained at about 50° C. A 500 ml high pressure vessel was used as vessel 508 in FIG. 5. Pressure of the system was maintained at 80 bar. The antisolvent flow consisted of 50 g/min of carbon dioxide. The solution flow rate was maintained at 0.5 mil/min for 100 minutes. A 100 micron capillary tube was used to disperse the solution inside the vessel M. After the end of solution flow, carbon dioxide flow was maintained for 60 additional minutes and the system was depressurized. The vessel was opened and the powder material was collected, weighed, labeled and stored.

### Example 20

Etoposide and paclitaxel was made into a solution in methanol and processed using process 1. Compressed carbon dioxide was used as the antisolvent. 0.5 g of etoposide and 0.5 g of paclitaxel were dissolved in 25 ml of methanol and dispensed in to the vessel. The temperature of the precipitation vessel was maintained at about 50° C. A 103 ml high pressure vessel was used as vessel 404 in FIG. 4. Pressure of the system was maintained at 100 bar. A magnetic stirrer at 520 rpm was used to obtain uniform mixing during the precipitation. The antisolvent flow consisted of 5 g/min of carbon dioxide. Pressure in the vessel was increased gradually at a rate of 1 bar/min. After the pressure reached 100 bar, antisolvent flow was maintained for 180 minutes. The vessel was depressurized, opened and the powder material was collected, weighed, labeled and stored.

### Example 21

Etoposide and paclitaxel was made into a solution in methanol and processed using process 1. Compressed carbon dioxide was used as the antisolvent. 1.0 g of etoposide and 0.5 g of paclitaxel were dissolved in 25 ml of methanol and dispensed in to the vessel. The temperature of the precipitation vessel was maintained at about 50° C. A 103 ml high pressure vessel was used as vessel 404 in FIG. 4. Pressure of the system was maintained at 100 bar. A magnetic stirrer at 520 rpm was used to obtain uniform mixing during the precipitation. The antisolvent flow consisted of 5 g/min of carbon dioxide. Pressure in the vessel was increased gradually at a rate of 1 bar/min. After the pressure reached 100 bar, antisolvent flow was maintained for 180 minutes. The vessel was depressurized, opened and the powder material was collected for further processing.

The samples produced by Examples 12-21 were tested for chemical integrity (using HPLC) and physical characteristics (using XRPD, DSC, TGA, SEM, NMR and IR).

HPLC analysis was carried out on each sample. Samples were dissolved in ethanol:methanol (50:50) to yield a concentration of 5 mg/mL. This stock solution was further diluted in ethanol:water (50:50) to yield a final concentration of 10 μg/mL.

Each drug showed a single peak with a retention time of about 6.5 and 8 minutes for etoposide and paclitaxel, respectively. The amount of recovery of individual drugs in each example was calculated based on the average of the experimentally determined concentration for three different samples prepared from each sample in Examples 1-10. The individual recoveries of etoposide and paclitaxel (including standard deviation (SD)) and combined drug recovery are set forth in Table 3.

TABLE 3
Concentrations of etoposide and paclitaxel (10 μg/mL samples)

| | Etoposide | | Paclitaxel | | Etopo- | Pacli- | |
|---|---|---|---|---|---|---|---|
| | Conc. | | Conc. | | side % | taxel % | |
| Ex- | (μg/mL) | | (μg/mL) | | of total | of total | Total % |
| ample | Average | SD | Average | SD | sample | sample | Recovery |
| 1 | 6.44 | 0.47 | 2.96 | 0.24 | 64.45 | 29.60 | 94.04 |
| 2 | 3.83 | 0.32 | 3.93 | 0.38 | 38.34 | 39.31 | 77.65 |
| 3 | 4.23 | 0.22 | 4.22 | 0.35 | 42.29 | 42.21 | 84.50 |
| 4 | 5.07 | 0.35 | 3.35 | 0.43 | 50.69 | 33.47 | 84.17 |
| 5 | 5.71 | 1.24 | 3.29 | 1.29 | 57.09 | 32.89 | 89.98 |
| 6 | 6.87 | 1.06 | 2.33 | 0.76 | 68.65 | 23.31 | 91.96 |
| 7 | 6.40 | 0.31 | 2.12 | 0.11 | 64.04 | 21.19 | 85.23 |
| 8 | 5.87 | 0.34 | 2.69 | 0.11 | 58.68 | 26.90 | 85.58 |
| 9 | 5.84 | 0.15 | 2.32 | 0.10 | 58.43 | 23.19 | 81.62 |
| 10 | 7.25 | 0.34 | 0.80 | 0.24 | 72.53 | 8.04 | 80.56 |

As Table 2 illustrates, drug recovery for the samples made in accordance with the present invention range from about 77% to about 94%. The overall average recovery for the samples of Examples 12-21 combined was 85.51±5.15.

SEM pictures of individual drugs show irregular particles and acicular (fibrous) clumps for paclitaxel and blade-like particles for etoposide. The SEM pictures of the combination samples show similarity in particle structure to etoposide and paclitaxel to varying extent. XRPD data shows the crystalline nature of the individual drugs and the samples prepared according to the invention with many of the samples similar to the individual drugs. DSC, IR and NMR data showed similarity between the samples of Examples 12-21 and the individual drugs.

The sample of Example 12 shows the closest similarity to the individual components when mixed together in a homogeneous blend. Example 12 provided the maximum recovery (based on HPLC data), appears to be a uniform blend of individual drugs (per SEM), and provides the closest IR spectra to the individual drugs. XRPD, DSC and NMR data also demonstrate that the sample of Example 12 is a homogenous, uniform blend of the individual etoposide and paclitaxel components.

As demonstrated by the results of Examples 12-21, the process of the invention can be used to produce pharmaceutical formulations comprising etoposide and paclitaxel by using a supercritical fluid in accordance with the present invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A process for preparing a pharmaceutical formulation containing two or more active pharmaceutical ingredients comprising:
(a) contacting two or more active pharmaceutical ingredients with a supercritical fluid to form a supercritical fluid solution; and
(b) separating the active ingredients from the supercritical fluid solution to yield a powder precipitate.

2. The process according to claim 1, wherein the supercritical fluid is carbon dioxide.

3. The process according to claim 2, wherein at least one of the active pharmaceutical ingredients is an anti-infective agent.

4. The process according to claim 3, wherein the anti-infective ingredient is selected from the group consisting of macrolide antibiotics, anthracycline antibiotics, quinolone antibiotics, cephalosporins, β-lactam antibiotics, penicillins, aminoglycosides, and sulfonamides.

5. The process according to claim 2, wherein the active pharmaceutical ingredients are a combination of two anti-infective agents.

6. The process according to claim 5, wherein the combination of two anti-infective agents are selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

7. The process according to claim 2, wherein at least one of the active pharmaceutical ingredients is an anticancer agent.

8. The process according to claim 7, wherein the anticancer agent is selected from the group consisting of etoposide, paclitaxel, altretamine, cisplatin, sarcolysine, alkylating agents, bleomycin, busulfan, docetaxel, carboplatin, doxorubicin, vincristine, fluorouracil, methotrexate, vinorelbine, cyclophosphamide/, ifosfarnide, mesna, gemcitabine hydrochloride, irinotecan hydrochloride, 5-fluorouracil, platinoids, and vinorelbine tartarate.

9. The process according to claim 2, wherein the active pharmaceutical ingredients are a combination of two anticancer agents.

10. The process according to claim 9, wherein the combination of two anticancer agents are selected from the group consisting of etoposide/paclitaxel, altretamine/cisplatin; altretamine/sarcolysine, altretamine/alkylating agents, bleomycin/cisplatin, busulfan/docetaxel, busulfan/carboplatin, cisplatin/doxorubicin, cisplatin/vincristine, cisplatin/fluorouracil, cisplatin/methotrexate, cisplatin/vinorelbine, cyclophosphamide/etoposide, etoposide/ifosfamide, ifosfamide/mesna, gemcitabine hydrochloride/cisplatin, gemcitabine/paclitaxel, irinotecan hydrochloride/5-fluorouracil, paclitaxel/platinoids, vinorelbine tartarate/platinoids, vinorelabine tartrate/paclitaxel, paclitaxel/cisplatin, and toposide/cisplatin.

11. The process according to claim 2, wherein the active ingredients are separated from the supercritical carbon dioxide solution by spraying the solution through a nozzle and recovering the precipitate.

12. The process according to claim 11, wherein the active pharmaceutical ingredients are contacted a solvent prior to step (a).

13. The process according to claim 11, wherein the supercritical fluid is contacted with a solvent prior to step (a).

14. A process of preparing a pharmaceutical formulation containing a combination of active pharmaceutical ingredients selected from the group consisting of two anti-infective agents and two anticancer agents comprising:
(a) contacting the two pharmaceutical ingredients with supercritical carbon dioxide to form a supercritical carbon dioxide solution;
(b) spraying the supercritical carbon dioxide solution through a nozzle; and
(c) recovering the precipitate in a powder form containing the combination of active pharmaceutical agents.

15. The process according to claim 14, wherein the combination of active pharmaceutical ingredients is a combination of anti-infective agents selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

16. The process according to claim 14, wherein the combination of active pharmaceutical ingredients is a combination of anticancer agents selected from the group consisting of etoposide/paclitaxel, altretamine/cisplatin; altretamine/sarcolysine, altretamine/alkylating agents, bleomycin/cisplatin, busulfan/docetaxel, busulfan/carboplatin, cisplatin/doxorubicin, cisplatin/vincristine, cisplatin/fluorouracil, cisplatin/methotrexate, cisplatin/vinorelbine, cyclophosphamide/etoposide, etoposide/ifosfamide, ifosfamide/mesna, gemcitabine hydrochloride/cisplatin, gemcitabine/paclitaxel, irinotecan hydrochloride/5-fluorouracil, paclitaxel/platinoids, vinorelbine tartarate/platinoids, vinorelabine tartrate/paclitaxel, paclitaxel/cisplatin, and toposide/cisplatin.

17. A process for preparing a pharmaceutical formulation containing two or more active pharmaceutical ingredients comprising:
(a) combining two or more active ingredients with a solvent to form a solution;
(b) contacting the solution with a supercritical fluid; and
(c) recovering the precipitate in a powder form.

18. The process according to claim 17, wherein the supercritical fluid is carbon dioxide.

19. The process according to claim 18, wherein at least one of the active pharmaceutical ingredients is an anti-infective agent.

20. The process according to claim 19, wherein the anti-infective agent is selected from the group consisting of macrolide antibiotics, anthracycline antibiotics, quinolone antibiotics, cephalosporins, β-lactam antibiotics, penicillins, aminoglycosides, and sulfonamides.

21. The process according to claim 18, wherein two or more of the active pharmaceutical ingredients are a combination of two anti-infective agents.

22. The process according to claim 21, wherein the combination of two anti-infective active ingredients are selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

23. The process according to claim 18, wherein the solution containing two or more active ingredients is contacted with the supercritical carbon dioxide by spraying the solution though a nozzle.

24. The process according to claim 18, wherein the solution containing two or more active ingredients is contacted with the supercritical carbon dioxide by pumping the supercritical carbon dioxide into the solution.

25. The process according to claim 18, wherein the supercritical carbon dioxide is contacted with a solvent prior to step (b).

26. A process for preparing a pharmaceutical formulation containing a combination of two active pharmaceutical ingredients selected from the group consisting of two anti-infective agents and two anticancer agents comprising anti-infective agents comprising:
(a) combining the two active pharmaceutical ingredients with a solvent to form a solution;
(b) contacting the solution with supercritical carbon dioxide; and
(c) recovering the precipitate in a powder form containing the combination of two active pharmaceutical ingredients.

27. The process according to claim 26, wherein the combination of two active pharmaceutical ingredients is a combination of two anti-infective agents selected from the group consisting of ampicillin sodium/sulbactam sodium, ticarcillin disodium/clavulanate potassium, quinupristin/dalfopristin, piperacillin sodium/tazobactam sodium, and imipenem/cilastatin.

28. The process according to claim 26, wherein the combination of active pharmaceutical ingredients is a combination of two anticancer agents selected from the group consisting of etoposide/paclitaxel, altretamine/cisplatin; altretamine/sarcolysine, altretamine/alkylating agents, bleomycin/cisplatin, busulfan/docetaxel, busulfan/carboplatin, cisplatin/doxorubicin, cisplatin/vincristine, cisplatin/fluorouracil, cisplatin/methotrexate, cisplatin/vinorelbine, cyclophosphamide/etoposide, etoposide/ifosfamide, ifosfamide/mesna, gemcitabine hydrochloride/cisplatin, gemcitabine/paclitaxel, irinotecan hydrochloride/5-fluorouracil, paclitaxel/platinoids, vinorelbine tartarate/platinoids, vinorelabine tartrate/paclitaxel, paclitaxel/cisplatin, and toposide/cisplatin.

29. The process according to claim 17, wherein the solution is contacted with a supercritical fluid by spraying the solution into a chamber containing the supercritical fluid.

30. The process according to claim 26, wherein the solution is contacted with a supercritical fluid by spraying the solution into a chamber containing the supercritical fluid.

31. A supercritical fluid solution comprising a supercritical fluid and two or more active pharmaceutical ingredients.

32. The supercritical fluid solution according to claim 31, wherein the supercritical fluid is supercritical carbon dioxide.

33. The supercritical fluid solution according to claim 32, wherein the solution contains two or more anti-infective active pharmaceutical ingredients.

34. The supercritical fluid solution according to claim 32, wherein the solution contains two or more anticancer active pharmaceutical ingredients.

35. The pharmaceutical formulation containing two or more active pharmaceutical ingredients selected from the group consisting of anti-infective agents and anticancer agents prepared by the process of claim 2.

36. A process for preparing a combination product containing two or more substances comprising:
(a) contacting two or more desired substances with a supercritical fluid to form a supercritical fluid solution; and
(b) separating the substances from the supercritical fluid solution to yield a powder precipitate.

37. A process for preparing a combination product containing two or more substances comprising:
(a) combining two or more substances with a solvent to form a solution;
(b) mixing the solution with a supercritical fluid; and
(c) recovering the precipitate in a powder form.
